(19) United States
(12) Patent Application Publication
(10) Pub. No.: US 2004/0019043 A1
(43) Pub. Date: Jan. 29, 2004

(54) Thiomolybdate analogues and uses thereof

(76) Inventors: Dimitri Coucouvanis, Ann Arbor, MI (US); Robert J. Ternansky, San Diego, CA (US); Patricia L. Gladstone, San Diego, CA (US); Amy L. Allan, Encinitas, CA (US); Melissa L.P. Price, San Deigo, CA (US); Sean M. O'Hare, San Diego, CA (US)

Correspondence Address: PENNIE AND EDMONDS, 1155 AVENUE OF THE AMERICAS, NEW YORK, NY, 100362711

(21) Appl. No.: 10/202,346

(22) Filed: Jul. 23, 2002

(51) Int. Cl. A61K 31/54; A61K 31/537; A61K 31/495; A61K 31/14; A61K 31/44

(52) U.S. Cl. 514/227.8; 514/231.2; 514/252.12; 514/358; 514/642; 514/643; 544/59; 544/106; 544/358; 546/347; 564/281; 564/282

(57) ABSTRACT

The current invention provides novel thiomolybdate derivatives, methods of making novel thiomolybdate derivatives, pharmaceutical compositions of novel thiomolybdate derivatives, methods of using novel thiomolybdate derivatives to treat diseases associated with aberrant vascularization and methods of using pharmaceutical compositions of thiomolybdate derivatives to treat diseases associated with aberrant vascularization.

### 1. FIELD OF THE INVENTION

The present invention relates generally to thiomolybdate derivatives, methods of making novel thiomolybdate derivatives, pharmaceutical compositions of novel thiomolybdate derivatives, methods of using novel thiomolybdate derivatives and pharmaceutical compositions of thiomolybdate derivatives to treat diseases associated with aberrant vascularization and/or excess levels of copper.

### 2. BACKGROUND OF THE INVENTION

Most forms of cancer are derived from solid tumors (Shockley et al., Ann. N. Y. Acad. Sci. 1991, 617: 367-382, which have proven resistant in the clinic to therapies such as the use of monoclonal antibodies and immunotoxins. Anti-angiogenic therapy for the treatment of cancer was developed from the recognition that solid tumors require angiogenesis (i.e., new blood vessel formation) for sustained growth (Folkman, Ann. Surg. 1972, 175: 409-416; Folkman, Mol. Med. 1995, 1(2): 120-122; Folkman, Breast Cancer Res. Treat. 1995, 36(2): 109-118; Hanahan et al., Cell 1996, 86(3): 353-364). Efficacy of anti-angiogenic therapy in animal models has been demonstrated (Millauer et al., Cancer Res. 1996, 56:1615-1620; Borgstrom et al., Prostrate 1998, 35:1-10; Benjamin et al., J. Clin. Invest. 1999, 103: 159-165; Merajver et al., Proceedings of Special AACR Conference on Angiogenesis and Cancer 1998, Abstract #B-11, January 22-24). In the absence of angiogenesis, internal cell layers of solid tumors are inadequately nourished. Further, angiogenesis (i.e., aberrant vascularization) has been implicated in numerous other diseases (e.g., ocular neovascular disease, macular degeneration, rheumatoid arthritis, etc.).

Contrastingly, normal tissue does not require angiogenesis except under specialized circumstances (e.g., wound repair, proliferation of the internal lining of the uterus during the menstrual cycle, etc.). Accordingly, a requirement for angiogenesis is a significant difference between tumor cells and normal tissue. Importantly, the dependency of tumor cells on angiogenesis, when compared to normal cells, is quantitatively greater than differences in cell replication and cell death, between normal tissue and tumor tissue, which are often exploited in cancer therapy.

Angiogenesis requires copper, as has been shown by numerous studies (Parke et al., Am. J. Pathol. 1988, 137:173-178; Raju et al., Natl. Cancer Inst. 1982, 69: 1183-1188; Ziche et al., Natl. Cancer Inst. 1982, 69: 475-482; Gullino, Anticancer Res. 1986, 6(2): 153-158). Attempts at preventing angiogenesis and hence tumor growth in animal models by reducing in vivo amounts of copper have been reported in the art (Brem et al., Neurosurgery 1990, 26:391-396; Brem et al., Am J. Pathol. 1990, 137(5): 1121-1142; Yoshida et al., Neurosurgery 1995 37(2): 287-295). These approaches incorporated both copper chelators and low copper diets.

More recently, Brewer et al., International Application No. PCT/US99/20374 have shown that the copper chelators, (e.g., tetrathiomolybdate) may be effective in treating diseases (e.g., solid tumor growth), which require angiogenesis. Accordingly, novel tetrathiomolybdate derivatives are required to fully explore the potential of thiomolybdate compounds in preventing angiogenesis. Such novel thiomolybdate derivatives may be effective treating various diseases associated with angiogenesis.

### 3. SUMMARY OF THE INVENTION

The present invention satisfies this and other needs by providing novel thiomolybdate derivatives, methods of making novel thiomolybdate derivatives, pharmaceutical compositions of novel thiomolybdate derivatives, methods of using novel thiomolybdate derivatives to treat diseases associated with aberrant vascularization and methods of using pharmaceutical compositions of thiomolybdate derivatives to treat diseases associated with aberrant vascularization or excess copper levels.

In a first aspect, the present invention provides a compound of structural formula (I):

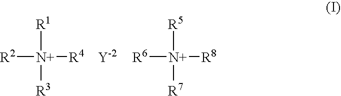

or a solvate or hydrate thereof wherein:

R¹, R², R³, R⁵, R⁶ and R⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl;

R⁴ and R⁸ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl or are absent when N is part of an aromatic ring;

optionally, R¹ and R² taken together are alkyldiyl, substituted alkyldiyl, heteroalkyldiyl or substituted heteroalkyldiyl;

optionally, R⁵ and R⁶ taken together are alkyldiyl, substituted alkyldiyl, heteroalkyldiyl or substituted heteroalkyldiyl;

optionally, R¹ and R² taken together, R² and R³ taken together and R² and R⁴ taken together are alkyldiyl, substituted alkyldiyl, heteroalkyldiyl or substituted heteroalkyldiyl;

optionally, R⁵ and R⁶ taken together, R⁶ and R⁷ taken together and R⁶ and R⁸ taken together are alkyldiyl, substituted alkyldiyl, heteroalkyldiyl or substituted heteroalkyldiyl;

optionally, R³ and R⁷ taken together are alkyldiyl, substituted alkyldiyl, heteroalkyldiyl or substituted heteroalkyldiyl; and

Y⁻² is (MoS₄)⁻², (Mo₂S₁₂)⁻², (Mo₂S₉)⁻², (Mo₂S₇)⁻², (Mo₂S₈)⁻², (Mo₂S₁₁)⁻², (Mo₂S₆)⁻² or (Mo₂S₁₃)⁻²;

with the proviso that if Y is (MoS₄)⁻² and R¹, R², R³³, R⁴, R⁵, R⁶, R⁷ and R⁸ are identical then each of R¹, R², R³, R⁴, R⁵, R⁶, R⁷ and R⁸ is not hydrogen, methyl, ethyl or n-propyl.

In a second aspect, the present invention provides pharmaceutical compositions of compounds of the invention. The pharmaceutical compositions generally comprise one or more compounds of the invention, pharmaceutically acceptable salts, hydrates or solvates thereof and a pharmaceutically acceptable diluent, carrier, excipient and adjuvant. The choice of diluent, carrier, excipient and adjuvant will depend upon, among other factors, the desired mode of administration.

In a third aspect, the present invention provides methods for treating or preventing diseases or disorders characterized by aberrant vascularization or aberrant angiogenesis or copper metabolism disorders. The methods generally involve administering to a patient in need of such treatment or prevention a therapeutically effective amount of a compound and/or pharmaceutical composition of the invention.

In a fourth aspect, the current invention provides pharmaceutical compositions for treating or preventing diseases or disorders characterized by aberrant vascularization or aberrant angiogenesis or copper metabolism disorders or excess copper levels in a patient in need of such treatment or prevention.

### 4. DETAILED DESCRIPTION OF THE INVENTION

4.1 Definitions

“Compounds of the invention” refers to compounds encompassed by structural formula (I) disclosed herein and includes any specific compounds within that generic formula whose structure is disclosed herein. The compounds of the invention may be identified either by their chemical structure and/or chemical name. When the chemical structure and chemical name conflict, the chemical structure is determinative of the identity of the compound. The compounds of the invention may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds of the invention may also exist in several tautomeric forms including the enol form, the keto form and mixtures thereof. Accordingly, the chemical structures depicted herein encompass all possible tautomeric forms of the illustrated compounds. The compounds of the invention also include isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, ³¹P, ³²P, ³⁵S, ¹⁸F, and ³⁶Cl. Further it should be understood, when partial structures of the compounds of the invention are illustrated, that brackets indicate the point of attachment of the partial structure to the rest of the molecule.

“Alkyl” refers to a saturated or unsaturated, branched, straight-chain or cyclic monovalent hydrocarbon group derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 20 carbon atoms, more preferably, from 1 to 10 carbon atoms.

“Alkanyl” refers to a saturated branched, straight-chain or cyclic alkyl group derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” refers to an unsaturated branched, straight-chain or cyclic alkyl group having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” refers to an unsaturated branched, straight-chain or cyclic alkyl group having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkyldiyl” refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Preferably, the alkyldiyl group is (C₁-C₂₀) alkyldiyl, more preferably, (C₁-C₂₀) alkyldiyl. Preferred are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkyleno, defined infra).

“Alkyleno” refers to a straight-chain alkyldiyl group having two terminal monovalent radical centers derived by the removal of one hydrogen atom from each of the two terminal carbon atoms of straight-chain parent alkane, alkene or alkyne. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, but[1,3]diyno, etc.; and the like. Where specific levels of saturation are intended, the nomenclature alkano, alkeno and/or alkyno is used. Preferably, the alkyleno group is (C₁-C₂₀) alkyleno, more preferably, (C₁-C₁₀) alkyleno. Preferred are straight-chain saturated alkano groups, e.g., methano, ethano, propano, butano, and the like.

“Acyl” refers to a radical —C(O)R, where R is hydrogen, alkyl, cycloalkyl, cycloheteroalkyl, aryl, arylalkyl, heteroalkyl, heteroaryl, heteroarylalkyl as defined herein. Representative examples include, but are not limited to formyl, acetyl, cyclohexylcarbonyl, cyclohexylmethylcarbonyl, benzoyl, benzylcarbonyl and the like.

“Acylamino” refers to a radical —NR′C(O)R, where R′ and R are each independently hydrogen, alkyl, cycloalkyl, cycloheteroalkyl, aryl, arylalkyl, heteroalkyl, heteroaryl, heteroarylalkyl, as defined herein. Representative examples include, but are not limited to, formylamino, acetylamino, cylcohexylcarbonylamino, cyclohexylmethyl-carbonylamino, benzoylamino, benzylcarbonylamino and the like.

“Alkylamino” means a radical —NHR where R represents an alkyl or cycloalkyl group as defined herein. Representative examples include, but are not limited to, methylamino, ethylamino, 1-methylethylamino, cyclohexylamino and the like.

“Alkoxy” refers to a radical —OR where R represents an alkyl or cycloalkyl group as defined herein. Representative examples include, but are not limited to, methoxy, ethoxy, propoxy, butoxy, cyclohexyloxy and the like.

“Alkoxycarbonyl” refers to a radical —C(O)-alkoxy where alkoxy is as defined herein.

“Alkylsulfonyl” refers to a radical —S(O)₂R where R is an alkyl or cycloalkyl group as defined herein. Representative examples include, but are not limited to methylsulfonyl, ethylsulfonyl, propylsulfonyl, butylsulfonyl and the like.

“Alkylsulfinyl” refers to a radical —S(O)R where R is an alkyl or cycloalkyl group as defined herein. Representative examples include, but are not limited to, methylsulfinyl, ethylsulfinyl, propylsulfinyl, butylsulfinyl and the like.

“Alkylthio” refers to a radical —SR where R is an alkyl or cycloalkyl group as defined herein that may be optionally substituted as defined herein. Representative examples include, but are not limited to methylthio, ethylthio, propylthio, butylthio, and the like.

“Amino” refers to the radical —NH₂.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Preferably, an aryl group comprises from 5 to 20 carbon atoms, more preferably from 5-12 carbon atoms.

“Arylalkyl” refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. Preferably, an arylalkyl group is (C₅-C₃₀) arylalkyl, e.g., the alkanyl, alkenyl or alkynyl moiety of the arylalkyl group is (C₁-C₁₀) and the aryl moiety is (C₆-C₂₀), more preferably, the arylalkyl group is (C₅-C₂₀) arylalkyl, e.g., the alkanyl, alkenyl or alkynyl moiety of the arylalkyl group is (C₁-C₁₀) and the aryl moiety is (C₅-C₁₂).

“Arylalkyloxy” refers to an —O-arylalkyl group where arylalkyl is as defined herein.

“Aryloxycarbonyl” refers to a radical —C(O)—O-aryl where aryl is as defined herein.

“Carbamoyl” refers to the radical —C(O)N(R)₂ where each R group is independently hydrogen, alkyl, cycloalkyl or aryl as defined herein, which may be optionally substituted as defined herein.

“Carboxy” means the radical —C(O)OH.

“Cyano” means the radical —CN.

“Cycloalkyl” refers to a saturated or unsaturated cyclic alkyl group. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In a preferred embodiment, the cycloalkyl group is (C₃-C₁₀) cycloalkyl, more preferably (C₃-C₇) cycloalkyl.

“Cycloheteroalkyl” refers to a saturated or unsaturated cyclic alkyl group in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, imidazolidine, morpholine, piperazine, piperidine, pyrazolidine, pyrrolidine, quinuclidine, and the like.

“Cycloheteroalkyloxycarbonyl” refers to a radical —C(O)—R where R is cycloheteroalkyl is as defined herein.

“Dialkylamino” means a radical —NRR′ where R and R′ independently represent an alkyl or cycloalkyl group as defined herein. Representative examples include, but are not limited to dimethylamino, methylethylamino, di-(1-methylethyl)amino, (cyclohexyl)(methyl)amino, (cyclohexyl)(ethyl)amino, (cyclohexyl)(propyl)amino, and the like.

“Halo” means fluoro, chloro, bromo, or iodo.

“Heteroalkyloxy” means an —O-heteroalkyl group where heteroalkyl is as defined herein.

“Heteroalkyl, Heteroalkanyl, Heteroalkenyl, Heteroalkanyl, Heteroalkyldiyl and Heteroalkyleno” refer to alkyl, alkanyl, alkenyl, alkynyl, alkyldiyl and alkyleno groups, respectively, in which one or more of the carbon atoms (and any associated hydrogen atoms) are each independently replaced with the same or different heteroatomic groups. Typical heteroatomic groups which can be included in these groups include, but are not limited to, —O—, —S—, —O—O—, —S—S—, —O—S—, —NR′—, ═N—N═, —N═N—, —N═N—NR′—, —PH—, —P(O)₂—, —O—P(O)₂—, —SH₂—, —S(O)₂—, —SnH₂— and the like, where each R′ is independently hydrogen, alkyl, aryl, arylaryl, arylalkyl, heteroaryl, heteroarylalkyl or heteroaryl-heteroaryl as defined herein.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-20 membered heteroaryl, with 5-10 membered heteroaryl being particularly preferred. Preferred heteroaryl groups are those derived from thiophene, pyrrole, benzothiophene, benzofuran, indole, pyridine, quinoline, imidazole, oxazole and pyrazine.

“Heteroaryloxycarbonyl” refers to a radical —C(O)—OR where R is heteroaryl as defined herein.

“Heteroarylalkyl” refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. Preferably, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20 membered heteroaryl, more preferably, the heteroarylalkyl group is a 6-20 membered heteroarylalkyl, e.g., the alkanyl, alkenyl or alkynyl moiety of the heteroarylalkyl is 1-10 membered and the heteroaryl moiety is a 5-10 membered heteroaryl.

“Hydroxy” means the radical —OH.

“Oxo” means the divalent radical═O.

“Substituted” refers to a group in which one or more hydrogen atoms are each independently replaced with the same or different substituent(s). Typical substituents include, but are not limited to, —X, —R¹⁴, —O⁻, ═O, —OR¹⁴, —SR¹⁴, —S⁻, ═S, —NR¹⁴R¹⁵, ═NR¹⁴, —CX₃, —CF₃, —CN, —OCN, —SCN, —NO, —NO₂, ═N₂, —N₃, —S(O)₂O⁻, —S(O)₂OH, —S(O)₂R¹⁴, —OS(O₂)O⁻, —OS(O)₂R¹⁴, —P(O)(O⁻)₂, —P(O)(OR¹⁴)(O⁻), —OP(O)(OR¹⁴)(OR¹⁵), —C(O)R¹⁴, —C(S)R¹⁴, —C(O)OR¹⁴, —C(O)NR¹⁴R¹⁵, —C(O)O⁻, —C(S)OR¹⁴, —NR¹⁶C(O)NR¹⁴R¹⁵, —NR¹⁶C(S)NR¹⁴R¹⁵, —NR¹⁷C(NR¹⁶)NR¹⁴R¹⁵ and —C(NR¹⁶)NR¹⁴R¹⁵, where each X is independently a halogen; each R¹⁴, 1R¹⁵, R¹⁶ and R¹⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR¹⁸R¹⁹, —C(O)R¹⁸ or —S(O)₂)₂R¹⁸ or optionally R¹⁸ and R¹⁹ together with the atom to which they are both attached form a cycloheteroalkyl or substituted cycloheteroalkyl ring; and R¹⁸ and R¹⁹ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Thio” means the radical —SH.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

4.2 Compounds of Structural Formula (I)

The compounds of the invention include compound of structural formula (I):

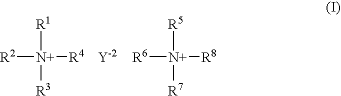

or a solvate or hydrate thereof wherein:

R¹, R², R³, R⁵, R⁶ and R⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl;

R⁴ and R⁸ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl or are absent when N is part of an aromatic ring;

optionally, R¹ and R² taken together are alkyldiyl, substituted alkyldiyl, heteroalkyldiyl or substituted heteroalkyldiyl;

optionally, R⁵ and R⁶ taken together are alkyldiyl, substituted alkyldiyl, heteroalkyldiyl or substituted heteroalkyldiyl;

optionally, R¹ and R² taken together, R² and R³ taken together and R² and R⁴ taken together are alkyldiyl, substituted alkyldiyl, heteroalkyldiyl or substituted heteroalkyldiyl;

optionally, R⁵ and R⁶ taken together, R⁶ and R⁷ taken together and R⁶ and R⁸ taken together are alkyldiyl, substituted alkyldiyl, heteroalkyldiyl or substituted heteroalkyldiyl;

optionally, R³ and R⁷ taken together are alkyldiyl, substituted alkyldiyl, heteroalkyldiyl or substituted heteroalkyldiyl; and

Y⁻² is (MoS₄)⁻², (Mo₂S₁₂)⁻², (Mo₂S₉)⁻², (Mo₂S₇)⁻², (Mo₂S₈)⁻², (Mo₂S₁₁)⁻², (Mo₂S₆)⁻² or (Mo₂S₁₃)⁻²;

with the proviso that if Y is (MoS₄)⁻² and R¹, R², R³, R⁴, R⁵, R⁶, R⁷ and R⁸ are identical then each of R¹, R², R³, R⁴, R⁵, R⁶, R⁷ and R⁸ is not hydrogen, methyl, ethyl or n-propyl.

In one preferred embodiment, Y is (MoS₄)⁻². Preferably, R¹, R², R³, R⁴, R⁵, R⁶, R⁷ and R⁸ are not alkyl.

In another preferred embodiment,

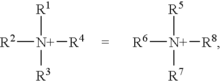

preferred embodiment, Y is (MoS₄)⁻².

In one embodiment, at least one of R¹, R², R³ and R⁴ is not alkyl. In another embodiment, R¹, R² and R⁴ are hydrogen, alkanyl or substituted alkanyl. Preferably, R¹, R² and R⁴ are hydrogen, methyl or ethyl.

In still another embodiment, R¹ and R² are alkanyl. Preferably, R¹ and R² are methyl or ethyl.

In still another embodiment, R¹ is alkanyl, substituted alkanyl, alkenyl, substituted alkenyl, aryl, substituted aryl, arylalkyl, substituted arylalkyl, cycloalkyl or substituted cycloalkyl. Preferably, R¹ and R² taken together are alkyleno, substituted alkyleno, heteroalkyleno or substituted heteroalkyleno. More preferably, R¹ and R² taken together are alkyleno or heteroalkyleno.

In still another embodiment, R¹ and R² taken together, R² and R³ taken together and R² and R⁴ taken together are alkyleno, substituted alkyleno, heteroalkyleno or substituted heteroalkyleno. Preferably, R¹ and R² taken together, R² and R³ taken together and R² and R⁴ taken together are alkyleno. Preferably, R¹(R²)(R³)(R⁴)N has the structure:

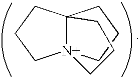

In still another embodiment, R³ and R⁷ taken together are alkyleno, substituted alkyleno, heteroalkyleno or substituted heteroalkyleno. Preferably, R³ and R⁷ taken together are alkyleno or heteroalkyleno.

In still another embodiment, R¹, R² and R⁴ are hydrogen, alkanyl or substituted alkanyl and R³ is alkyl, substituted alkyl, alkenyl, aryl, arylalkyl, cycloalkyl or R³ and R⁷ taken together are alkyleno, substituted alkyleno, heteroalkyleno or substituted heteroalkyleno. Preferably, R¹, R² and R⁴ are methyl or ethyl and R³ is alkyl, substituted alkyl, alkenyl, aryl, arylalkyl, cycloalkyl or R³ and R⁷ taken together are alkyleno or heteroalkyleno. Preferably, R¹, R² and R⁴ are methyl or ethyl and R³ is alkyl, substituted alkyl, alkenyl, aryl, arylalkyl or cycloalkyl.

In still another embodiment, R¹(R²)(R³)(R⁴)N is

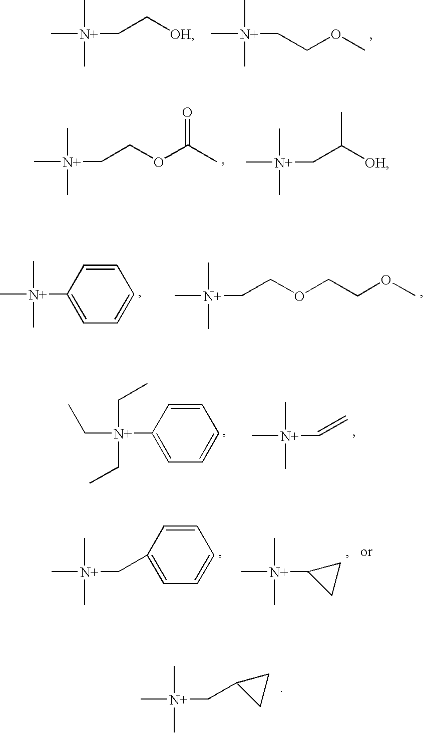

In still another embodiment, R¹(R²)(R³)(R⁴)N is

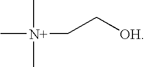

In still another embodiment, R¹(R²)(R³)(R⁴)N is

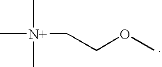

In still another embodiment, R¹(R²)(R³)(R⁴)N is

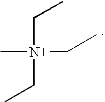

In still another embodiment, R¹, R² and R⁴ are methyl or ethyl and R³ and R⁷ taken together are alkyleno or heteroalkyleno. Preferably, R¹(R²)(R³)(R⁴)N has the structure:

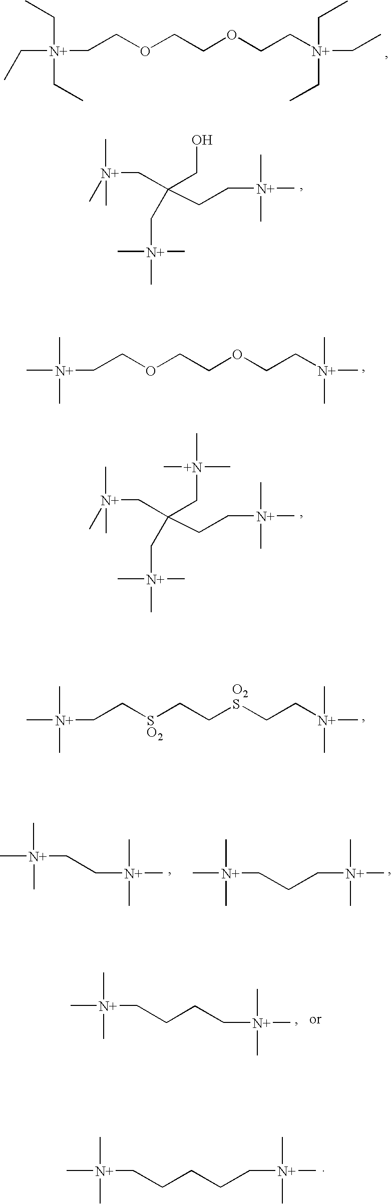

In still another embodiment, R¹, R² and R⁴ are hydrogen and R³ is substituted alkyl, cycloalkyl or substituted heteroaryl or R³ and R⁷ taken together are alkyleno. In still another embodiment, R¹ and R² are alkanyl and R³ and R⁴ are alkyl, substituted alkyl, aryl, arylalkyl or alkyleno. Preferably, R¹ and R² are methyl or ethyl and R³ and R⁴ are alkyl, substituted alkyl, aryl, arylalkyl or alkyleno.

In still another embodiment, R¹(R²)(R³)(R⁴)N are

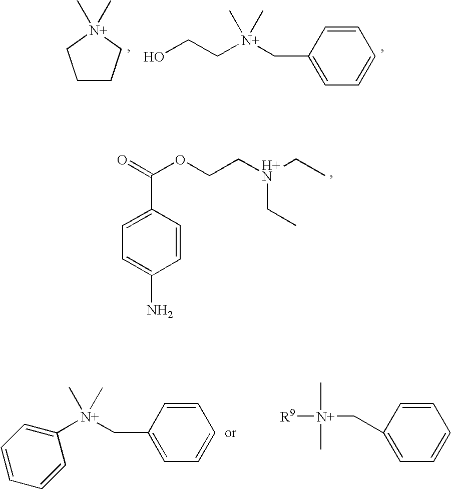

wherein R⁹ is a mixture of straight chain alkanyl groups which have at least eight carbon atoms and not more than eighteen carbon atoms.

In still another embodiment, R¹, R² and R⁴ are hydrogen and R³ is substituted alkyl, substituted heteroaryl, cycloalkyl or alkyleno. Preferably, R¹(R²)(R³)(R⁴)N has the structure:

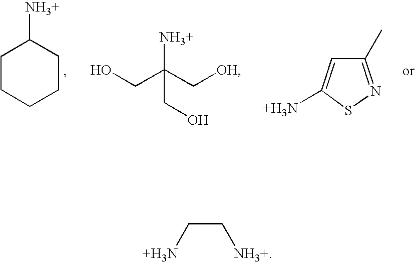

In still another embodiment, R¹ and R² taken together are alkyleno, substituted alkyleno, heteroalkyleno or substituted heteroalkyleno, R³ is alkyl or substituted alkyl and R⁴ is hydrogen or is absent. Preferably, R¹(R²)(R³)N or R¹(R²)(R³)(R⁴)N has the structure:

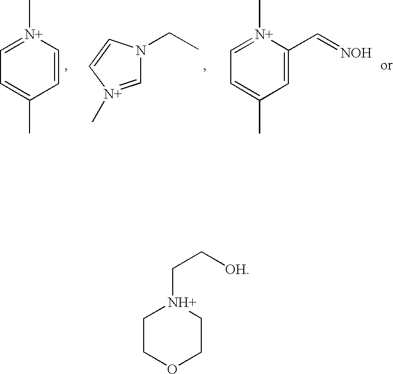

4.3 Synthesis of the Compounds of the Invention

The compounds of the invention may be obtained via conventional synthetic methods illustrated in Schemes 1 and 2. Starting materials useful for preparing compounds of the invention and intermediates thereof are commercially available or can be prepared by well-known synthetic methods. For example, ammonium thiomolybdate may be purchased from well-known chemical suppliers (e.g., Aldrich Chemical Company, Milwaukee, Wis.). Substituted ammonium salts (e.g., ammonium hydroxide and ammonium halides) may be either purchased from commercial sources or may be readily synthesized using well-known synthetic methods (Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995). Other methods for synthesis of the compounds described herein and/or starting materials are either described in the art or will be readily apparent to the skilled artisan. Accordingly, the methods presented in Schemes 1 and 2 herein are illustrative rather than comprehensive.

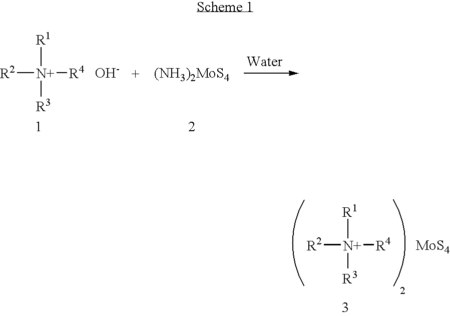

As shown above, in Scheme 1, addition of a quaternary ammonium hydroxide to thiomolybdate in the presence of water leads to cation exchange (equilibrium to product is driven by removal of volatile ammonia) to provide the desired thiomolybdate derivative.

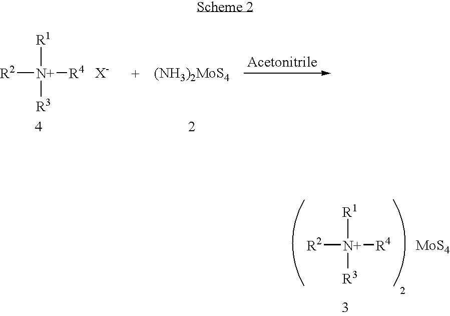

As shown above, in Scheme 2, addition of a quaternary ammonium halide to thiomolybdate in the presence of acetonitrile leads to cation exchange (equilibrium to product is driven by formation of ammonium halide) to provide the desired thiomolybdate derivative.

It should be noted that thiomolybdate derivatives where the ammonium counterions are different may be prepared from compounds 3 through by treating with one equivalent of a different ammonium counterion. Such a reaction would be expected to produce a statistical mixture of products.

4.4 Therapeutic Uses of the Compounds of Structural Formula (I)

In accordance with the invention, a compound of structural formula (I) and/or a pharmaceutical composition thereof is administered to a patient, preferably a human, suffering from a disease characterized by aberrant vascularization or aberrant angiogenesis. Aberrant vascularization or aberrant angiogenesis includes abnormal neovascularization such as the formation of new blood vessels, larger blood vessels, more branched blood vessels and any other mechanism, which inappropriate or increased blood carrying capacity to a diseased tissue or site. The compounds of the invention and pharmaceutical compositions of the invention treat or prevent aberrant vascularization or aberrant angiogenesis.

Preferably, diseases characterized by aberrant vascularization or aberrant angiogenesis include cancer (e.g., any vascularized tumor, preferably, a solid tumor, including but not limited to, carcinomas of the lung, breast, ovary, stomach, pancreas, larynx, esophagus, testes, liver, parotid, bilary tract, colon, rectum, cervix, uterus, endometrium, kidney, bladder, prostrate, thyroid, squamous cell carcinomas, adenocarcinomas, small cell carcinomas, melanomas, gliomas, neuroblastomas, sarcomas (e.g., angiosarcomas, chondrosarcomas)), arthritis, diabetes, arteriosclerosis, arteriovenous, malformations, corneal graft neovascularization, delayed wound healing, diabetic retinopathy, age related macular degeneration, granulations, burns, hemophilic joints, rheumatoid arthritis, hypertrophic scars, neovascular gluacoma, nonunion fractures, Osier Weber Syndrome, Psoriasis, pyogenic, granuloma, retrolental fibroplasia, pterygium, scleroderma, trachoma, vascular adhesions, ocular neovascularization, parasitic diseases, hypertrophy following surgery, inhibition of hair growth, macular degeneration (including both wet and dry type), rheumatoid arthritis and osteoarthritis. More preferably, diseases characterized by aberrant vascularization and aberrant angiogenesis include cancer, macular degeneration and rheumatoid arthritis.

Further, in accordance with the invention, a compound of structural formula (I) and/or a pharmaceutical composition thereof is administered to a patient, preferably a human, suffering from a disease associated with copper metabolism disorders (e.g., Wilson's disease).

Further, in certain embodiments, a compounds of the invention and and/or pharmaceutical compositions thereof are administered to a patient, preferably a human, as a preventative measure against various diseases or disorders characterized by aberrant vascularization or aberrant angiogenesis or copper metabolism disorders. Thus, the compounds of structural Formula (I) and/or pharmaceutical compositions thereof may be administered as a preventative measure to a patient having a predisposition for a disease characterized by aberrant vascularization or aberrant angiogenesis or copper metabolism disorders. Accordingly, the compounds of structural Formulae (I) and/or pharmaceutical compositions thereof may be used for the prevention of one disease or disorder and concurrently treating another (e.g., preventing Wilson's disease while treating cancer).

The suitability of the compounds of structural Formula (I) and/or pharmaceutical compositions of compounds of Formula (I) in treating or preventing various diseases or disorders characterized by aberrant vascularization or aberrant angiogenesis or copper metabolism disorders may be determined by methods described in the art. Accordingly, it is well with the capability of those of skill in the art to assay and use the compounds of structural Formulae (I) and/or pharmaceutical compositions thereof to treat.

4.5 Therapeutic/Prophylactic Administration

The compounds and/or pharmaceutical compositions of the invention may be advantageously used in human medicine. As previously described in Section 4.5 above, compounds of structural Formula (I) and/or pharmaceutical compositions thereof are useful for the treatment or prevention of various diseases or disorders characterized by aberrant vascularization or aberrant angiogenesis or copper metabolism disorders.

When used to treat or prevent the above disease or disorders, compounds and/or pharmaceutical compositions of the invention may be administered or applied singly, or in combination with other agents. The compounds and/or pharmaceutical compositions of the invention may also be administered or applied singly, in combination with other pharmaceutically active agents (e.g., other anti-cancer agents, other anti-angiogenic agents and other chelators such as zinc, penicillamine, etc.), including other compounds of the invention.

The current invention provides methods of treatment and prophylaxis by administration to a patient of a therapeutically effective amount of a pharmaceutical composition or compound of the invention. The patient may be an animal, is more preferably a mammal, and most preferably a human.

The present compounds and/or pharmaceutical compositions of the invention, which comprise one or more compounds of the invention, are preferably administered orally. The compounds and/or pharmaceutical compositions of the invention may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound and/or pharmaceutical composition of the invention. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the compounds and/or pharmaceutical compositions of the invention into the bloodstream.

In specific embodiments, it may be desirable to administer one or more compounds and/or pharmaceutical composition of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of cancer or arthritis.

In certain embodiments, it may be desirable to introduce one or more compounds and/or pharmaceutical compositions of the invention into the central nervous system by any suitable route, including intraventricular, intrathecal and epidural injection. Intraventricular injection may be facilitated by an intraventricular catheter, for example, attached to a reservoir, such as an Ommaya reservoir.

A compound and/or pharmaceutical composition of the invention may also be administered directly to the lung by inhalation. For administration by inhalation, a compound and/or pharmaceutical composition of the invention may be conveniently delivered to the lung by a number of different devices. For example, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver compounds of the invention directly to the lung.

Alternatively, a Dry Powder Inhaler (“DPI”) device may be used to administer a compound and/or pharmaceutical composition of the invention to the lung. DPI devices typically use a mechanism such as a burst of gas to create a cloud of dry powder inside a container, which may then be inhaled by the patient. DPI devices are also well known in the art. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator may be formulated containing a powder mix of a compound of the invention and a suitable powder base such as lactose or starch for these systems.

Another type of device that may be used to deliver a compound and/or pharmaceutical composition of the invention to the lung is a liquid spray device supplied, for example, by Aradigm Corporation. Liquid spray systems use extremely small nozzle holes to aerosolize liquid drug formulations that may then be directly inhaled into the lung.

In one embodiment, a nebulizer is used to deliver a compound and/or pharmaceutical composition of the invention to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96, which is herein incorporated by reference). Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974), Aventis and Batelle Pulmonary Therapeutics.

In another embodiment, an electrohydrodynamic (“EHD”) aerosol device is used to deliver a compound and/or pharmaceutical composition of the invention to the lung. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering a compound and/or pharmaceutical composition of the invention to the lung with an EHD aerosol device and such optimization is routinely performed by one of skill in the art. EHD aerosol devices may more efficiently deliver drugs to the lung than existing pulmonary delivery technologies.

In another embodiment, the compounds and/or pharmaceutical compositions of the invention can be delivered in a vesicle, in particular a liposome (see Langer, 1990, Science, 249:1527-1533; Treat et al, in “Liposomes in the Therapy of Infectious Disease and Cancer,” Lopez-Berestein and Fidler (eds.), Liss, New York, pp.353-365 (1989); see generally “Liposomes in the Therapy of Infectious Disease and Cancer,” Lopez-Berestein and Fidler (eds.), Liss, New York, pp.353-365 (1989)).

In another embodiment, the compounds and/or pharmaceutical compositions of the invention can be delivered via sustained release systems, preferably oral sustained release systems. In one embodiment, a pump may be used (see Langer, supra; Sefton, 1987, CRC Crit Ref Biomed Eng. 14:201; Saudek et al., 1989, N. Engl. J Med. 321:574).

In another embodiment, polymeric materials can be used (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al., 1989, J. Neurosurg. 71:105). In another embodiment, polymeric materials are used for oral sustained release delivery. Preferred polymers include sodium carboxymethylcellulose, hydroxypropylcellulose, hydroxypropylmethylcellulose and hydroxyethylcellulose (most preferred, hydroxypropyl methylcellulose). Other preferred cellulose ethers have been described (Alderman, Int. J. Phann. Tech. & Prod. Mfr., 1984, 5(3) 1-9). Factors affecting drug release are well known to the skilled artisan and have been described in the art (Bamba et al., Int. J. Pharm., 1979, 2, 307).

In another embodiment, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In still another embodiment, osmotic delivery systems are used for oral sustained release administration (Verma et al., Drug Dev. Ind. Pharm., 2000, 26:695-708). In another embodiment, OROS™ osmotic devices are used for oral sustained release delivery devices (Theeuwes et al., U.S. Pat. No. 3,845,770; Theeuwes et al., U.S. Pat. No. 3,916,899).

In yet another embodiment, a controlled-release system can be placed in proximity of the target of the compounds and/or pharmaceutical composition of the invention, thus requiring only a fraction of the systemic dose (see, e.g., Goodson, in “Medical Applications of Controlled Release,” supra, vol. 2, pp. 115-138 (1984)). Other controlled-release systems discussed in Langer, 1990, Science 249:1527-1533 may also be used.

4.6 Pharmaceutical Compositions of the Invention

The present pharmaceutical compositions contain a therapeutically effective amount of one or more compounds of the invention, preferably in purified form, together with a suitable amount of a pharmaceutically acceptable vehicle, which so as to provide the form for proper administration to a patient. When administered to a patient, the compounds of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions comprising a compound of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds of the invention into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 17th Edition, 1985).

For topical administration a compound of the invention may be formulated as solutions, gels, ointments, creams, suspensions, etc. as is well-known in the art. Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

In a preferred embodiment, the compounds of the invention are formulated in accordance with routine procedures as a pharmaceutical composition adapted for intravenous administration to human beings. Typically, compounds of the invention for intravenous administration are solutions in sterile isotonic aqueous buffer. For injection, a compound of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. When necessary, the pharmaceutical compositions may also include a solubilizing agent. Pharmaceutical compositions for intravenous administration may optionally include a local anesthetic such as lignocaine to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. When the compound of the invention is administered by infusion, it can be dispensed, for example, with an infusion bottle containing sterile pharmaceutical grade water or saline. When the compound of the invention is administered by injection, an ampoule of sterile water for injection or saline can be provided so that the ingredients may be mixed prior to administration.

For transmucosal administration, penetrants appropriate to the barrier to be permeated are used in the formulation. Such penetrants are generally known in the art.

Pharmaceutical compositions for oral delivery may be in the form of tablets, lozenges, aqueous or oily suspensions, granules, powders, emulsions, capsules, syrups, or elixirs, for example. Orally administered pharmaceutical compositions may contain one or more optionally agents, for example, sweetening agents such as fructose, aspartame or saccharin; flavoring agents such as peppermint, oil of wintergreen, or cherry coloring agents and preserving agents, to provide a pharmaceutically palatable preparation. Moreover, where in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5.0 mM to about 50.0 mM) etc. Additionally, flavoring agents, preservatives, coloring agents, bile salts, acylcarnitines and the like may be added.

For buccal administration, the pharmaceutical compositions may take the form of tablets, lozenges, etc. formulated in conventional manner.

Liquid drug formulations suitable for use with nebulizers and liquid spray devices and EHD aerosol devices will typically include a compound of the invention with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the invention. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

A compound of the invention may also be formulated in rectal or vaginal pharmaceutical compositions such as suppositories or retention enemas, e.g., containing conventional suppository bases such as cocoa butter or other glycerides.

In addition to the formulations described previously, a compound of the invention may also be formulated as a depot preparation. Such long acting formulations may be administered by implantation (for example subcutaneously or intramuscularly) or by intramuscular injection. Thus, for example, a compound of the invention may be formulated with suitable polymeric or hydrophobic materials (for example as an emulsion in an acceptable oil) or ion exchange resins, or as sparingly soluble derivatives, for example, as a sparingly soluble salt.

When a compound of the invention is acidic, it may be included in any of the above-described formulations as the free acid, a pharmaceutically acceptable salt, a solvate or hydrate. Pharmaceutically acceptable salts substantially retain the activity of the free acid, may be prepared by reaction with bases and tend to be more soluble in aqueous and other protic solvents than the corresponding free acid form.

4.7 Methods of Use and Doses

A compound of the invention, or pharmaceutical compositions thereof, will generally be used in an amount effective to achieve the intended purpose. For use to treat or prevent diseases or disorders characterized by aberrant vascularization or aberrant angiogenesis or copper metabolism disorders the compounds of structural Formula (I) and/or pharmaceutical compositions thereof, are administered or applied in a therapeutically effective amount.

The amount of a compound of the invention that will be effective in the treatment of a particular disorder or condition disclosed herein will depend on the nature of the disorder or condition, and can be determined by standard clinical techniques known in the art as previously described. In addition, in vitro or in vivo assays may optionally be employed to help identify optimal dosage ranges. The amount of a compound of the invention administered will, of course, be dependent on, among other factors, the subject being treated, the weight of the subject, the severity of the affliction, the manner of administration and the judgment of the prescribing physician.

For example, the dosage may be delivered in a pharmaceutical composition by a single administration, by multiple applications or controlled release. In one embodiment, the compounds of the invention are delivered by oral sustained release administration. Preferably, in this embodiment, the compounds of the invention are administered twice per day (more preferably, once per day). Dosing may be repeated intermittently, may be provided alone or in combination with other drugs and may continue as long as required for effective treatment of the disease state or disorder.

Suitable dosage ranges for oral administration are dependent on the potency of the drug, but are generally about 0.001 mg to about 200 mg of a compound of the invention per kilogram body weight. Dosage ranges may be readily determined by methods known to the artisan of ordinary skill.

Suitable dosage ranges for intravenous (i.v.) administration are about 0.01 mg to about 100 mg per kilogram body weight. Suitable dosage ranges for intranasal administration are generally about 0.01 mg/kg body weight to about 1 mg/kg body weight. Suppositories generally contain about 0.01 milligram to about 50 milligrams of a compound of the invention per kilogram body weight and comprise active ingredient in the range of about 0.5% to about 10% by weight. Recommended dosages for intradermal, intramuscular, intraperitoneal, subcutaneous, epidural, sublingual or intracerebral administration are in the range of about 0.001 mg to about 200 mg per kilogram of body weight. Effective doses may be extrapolated from dose-response curves derived from in vitro or animal model test systems. Such animal models and systems are well-known in the art.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred for reducing convulsion. The compounds of the invention may also be demonstrated to be effective and safe using animal model systems.

Preferably, a therapeutically effective dose of a compound of the invention described herein will provide therapeutic benefit without causing substantial toxicity. Toxicity of compounds of the invention may be determined using standard pharmaceutical procedures and may be readily ascertained by the skilled artisan. The dose ratio between toxic and therapeutic effect is the therapeutic index. A compound of the invention will preferably exhibit particularly high therapeutic indices in treating disease and disorders. The dosage of a compound of the inventions described herein will preferably be within a range of circulating concentrations that include an effective dose with little or no toxicity.

4.8 Combination Therapy

In certain embodiments of the present invention, the compounds and/or pharmaceutical compositions of the invention can be used in combination therapy with at least one other therapeutic agent. The compound and/or pharmaceutical composition of the invention and the therapeutic agent can act additively or, more preferably, synergistically. In a preferred embodiment, a compound of the invention or a pharmaceutical composition of a compound of the invention is administered concurrently with the administration of another therapeutic agent, which may be part of the same pharmaceutical composition as the compound of the invention or a different pharmaceutical composition. In another embodiment, a pharmaceutical composition of a compound of the invention is administered prior or subsequent to administration of another therapeutic agent.

In particular, in one preferred embodiment, the compounds and/or pharmaceutical compositions of the invention can be used in combination therapy with other chemotherapeutic agents (e.g., alkylating agents (e.g., nitrogen mustards (e.g., cyclophosphamide, ifosfamide, mechlorethamine, melphalen, chlorambucil, hexamethylmelamine, thiotepa), alkyl sulfonates (e.g., busulfan), nitrosoureas, triazines) antimetabolites (e.g., folic acid analogs, pyrimidine analogs (e.g., fluorouracil, floxuridine, cytosine arabinoside, etc.), purine analogs (e.g., mercaptopurine, thiogunaine, pentostatin, etc.), natural products (e.g., vinblastine, vincristine, etoposide, tertiposide, dactinomycin, daunorubicin, doxurubicin, bleomycin, mithrmycin, mitomycin C, L-asparaginase, interferon alpha), platinum coordination complexes (e.g., cis-platinum, carboplatin, etc.), mitoxantrone, hydroxyurea, procarbazine, hormones and antagonists (e.g., prednisone, hydroxyprogesterone caproate, medroxyprogesterone acetate, megestrol acetate, diethylstilbestrol, ethinyl estradiol, tamoxifen, testosterone propionate, fluoxymesterone, flutamide, leuprolide, etc.), anti-angiogenesis agents or inhibitors (e.g., angiostatin, retinoic acids and paclitaxel, estradiol derivatives, thiazolopyrimidine derivatives, etc.), apoptosis-inducing agents (e.g., antisense nucleotides that block oncogenes which inhibit apoptosis, tumor suppressors , TRAIL, TRAIL polypeptide, Fas-associated factor 1, interleukin-1β-converting enzyme, phosphotyrosine inhibitors, RXR retinoid receptor agonists, carbostyril derivatives, etc.) and chelators (penicillamine, zinc, trientine, etc.)). Preferably, the compounds and/or pharmaceutical compositions of the invention are used in combination therapy with zinc.

4.9 Therapeutic Kits

The current invention provides therapeutic kits comprising the compounds of the invention or pharmaceutical compositions of the invention. The therapeutic kits may also contain other compounds (e.g., chemotherapeutic agents, natural products, hormones or antagonists, anti-angiogenesis agents or inhibitors, apoptosis-inducing agents or chelators) or pharmaceutical compositions of these other compounds.

Therapeutic kits may have a single containers which contains the compound of the invention or pharmaceutical compositions of the invention with or without other components (e.g., other compounds or pharmaceutical compositions of these other compounds) or may have distinct container for each component. Preferably, therapeutic kits of the invention include a compound of the invention or a pharmaceutical composition of the invention packaged for use in combination with the co-administration of a second compound (preferably, a chemotherapeutic agent, a natural product, a hormone or antagonist, a anti-angiogenesis agent or inhibitor, a apoptosis-inducing agent or a chelator) or a pharmaceutical composition thereof. The components of the kit may be pre-complexed or each component may be in a separate distinct container prior to administration to a patient.

The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container.

The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid.

Preferably, a therapeutic kit will contain apparatus (e.g., one or more needles, syringes, eye droppers, pipette, etc.), which enables administration of the components of the kit.

### 5. EXAMPLES

The invention is further defined by reference to the following examples, which describe in detail, preparation of compounds of the invention and methods for assaying for biological activity. It will be apparent to those skilled in the art that many modifications, both to materials and methods, may be practiced without departing from the scope of the invention.

### 5.1 Example 1

General Procedure for Synthesis of Tetrathiomolybdate Derivatives

The commercially available aqueous solution of quaternary ammonium hydroxide (2 eq.) was added to ammonium tetrathiomolybdate (1 eq.) followed by deionized water until all the solid material was dissolved. The solution was placed on a rotary evaporator under vacuum (ca. 5-10 torr) at 20° C. for 2 hours and water was replaced as needed to maintain a constant volume. If this procedure resulted in a precipitate, the solid was collected by filtration, washed with isopropanol, ethanol, and diethyl ether, and then dried under high vacuum for 24 hours in a vacuum desiccator in the presence of P₂O₅. If the solution remained clear, the reaction mixture was first filtered to remove small amounts of solid impurities, and the product was precipitated from the filtrate with isopropanol. The solid was collected by filtration, washed with isopropanol, ethanol, and diethyl ether and then dried under high vacuum for 24 hours in a vacuum desiccator in the presence of P₂O₅.

### 5.2 Example 2

General Procedure for Synthesis of Tetrathiomolybdate Derivatives

The solid quaternary ammonium halide (2 eq.) was added to a suspension of ammonium tetrathiomolybdate (1 eq.) in dry acetonitrile (5 mL per mmol of TM) and the resulting mixture was stirred at room temperature under nitrogen for 18 hours. If this procedure resulted in a precipitate, the solid was collected by filtration, washed with water, isopropanol, ethanol and diethyl ether, and then dried under high vacuum for 24 hours in a vacuum desiccator in the presence of P₂O₅. If the solution remained clear, the reaction mixture was first filtered, and the filtrate was concentrated in vacuo. The resulting solid was suspended in water and filtered, and the solid was washed with isopropanol, ethanol and diethyl ether and then dried under high vacuum for 24 hours in a vacuum desiccator in the presence of P₂O₅.

### 5.3 Example 3

Tetrathiomolybdate, bis(triethylmethyl ammonium)

This compound was prepared from ammonium tetrathiomolybdate (994 mg, 3.82 mmol) and a 20% by weight aqueous solution of triethylmethylammonium hydroxide (5.12 g, 7.69 mmol) according to the procedure of Example 1 and provided 1.05 g (60%) of the title compound as an orange-red solid. IR (KBr, cm⁻¹) 472; ¹H NMR (300 MHz, DMSO-d6) δ3.27 (q, J=7.3 Hz, 12H), 2.89 (s, 6H), 1.19 (tt, J=7.3, 1.8 Hz, 18H); ¹³C NMR (75 MHz, DMSO-d6) δ55.1 (6C), 46.1 (2C), 7.7 (6C); ES MS m/z (triethylmethyl ammonium)⁺ 116.3; UV (H₂O) 468 nm (ε=12000). Analysis: Calcd for C₁₄H₃₆MoN₂S₄: C, 36.82; H, 7.95; N, 6.13. Found: C, 37.07; H, 7.88; N, 6.24.

### 5.4 Example 4

Tetrathiomolybdate, bis(triethylphenyl ammonium)

This compound was prepared from ammonium tetrathiomolybdate (1.00 g, 3.85 mmol) and a 10% by weight aqueous solution of triethylphenylammonium hydroxide (15.1 g, 7.71 mmol) according to the procedure of Example 1, and provided 388 mg (17%) of the title compound as an orange solid. IR (KBr, cm⁻¹) 470; ¹H NMR (300 MHz, DMSO-d6) δ7.91 (d, J=8.2 Hz, 4H), 7.69-7.55 (m, 6H), 3.89 (q, J=6.9 Hz, 12H), 1.04 (t, J=6.9 Hz, 18H); ¹³C NMR (75 MHz, DMSO-d6) δ141.9 (2C), 130.5 (4C), 130.1 (2C), 122.7 (4C), 55.3 (6C), 8.0 (6C); ES MS m/z (triethylphenyl ammonium)⁺ 178.2; UV (H₂O) 468 nm (ε=12300). Analysis: Calcd for C₂₄H₄₀MoN₂S₄: C, 49.63; H, 6.94; N, 4.82; S, 22.08. Found: C, 49.39; H, 7.23; N, 5.15; S, 22.41.

### 5.5 Example 5

Tetrathiomolybdate, bis(choline)

In the instant application, bis(choline) tetrathiomolybdate has the same chemical structure as bis[2-hydroxyethyl)trimethyl ammonium] tetrathiomolybdate. This compound was prepared from ammonium tetrathiomolybdate (1.08 g, 4.15 mmol) and a 50% by weight aqueous solution of choline hydroxide (2.01 g, 8.29 mmol) according to the procedure of Example 1 and afforded 1.30 g (73%) of the title compound as an orange solid. IR (KBr, cm⁻¹) 3389, 474; ¹H NMR (300 MHz, DMSO-d6) δ5.21 (t, J=4.6 Hz, 2H), 3.87-3.79 (m, 4H), 3.43 (t, J=4.6 Hz, 4H), 3.13 (s, 18H); ¹³C NMR (75 MHz, DMSO-d6) δ67.1 (2C), 55.5 (2C), 53.3 (6C); ES MS m/z (choline)⁺ 104.2; UV (H₂O) 468 nm (ε=12900). Analysis: Calcd for C₁₀H₂₈MoN₂O₂S₄: C, 27.77; H, 6.52; N, 6.48; S, 29.65; Mo, 22.18. Found: C, 27.63; H, 6.84; N, 6.39; S, 29.86; Mo, 22.23.

### 5.6 Example 6

Tetrathiomolybdate, bis(acetylcholine)

This compound was prepared from acetylcholine chloride (732 mg, 4.03 mmol) and ammonium tetrathiomolybdate (500 mg, 1.92 mmol) according to the procedure of Example 2, and afforded 390 mg (39%) of the title compound as an orange-red solid. IR (KBr, cm⁻¹) 1747, 1728, 482, 469, 461; ¹H NMR (300 MHz, DMSO-d6) δ4.47-4.41 (m, 4H), 3.72-3.69 (m, 4H), 3.16 (s, 18H), 2.07 (s, 6H); ¹³C NMR (75 MHz, DMSO-d6) δ170.1 (2C), 63.9 (2C), 58.0 (2C), 53.1 (6C), 20.9 (2C); ES MS m/z (acetylcholine)⁺ 146.4; UV (H₂O) 468 nm (ε=13300). Analysis: Calcd for C₁₄H₃₂MoN₂O₄S₄: C, 32.55; H, 6.24; N, 5.42; S, 24.82. Found: C, 32.66; H, 6.23; N, 5.51; S, 24.97.

### 5.7 Example 7

Tetrathiomolybdate, bis[2-(methoxy)ethyltrimethyl ammonium]

This compound was prepared from ammonium tetrathiomolybdate (5 g, 19.2 mmol) and 2-(methoxy)ethyltrimethylammonium chloride (6.199 g, 40.3 mmol, see below in this example for synthesis) in a stirring suspension of acetonitrile (125 ml). The suspension was stirred for 2 hours, during which time a fine light red powder formed. The precipitate was filtered over a glass frit, and the acetonitrile was evaporated to afford 1.76 g (20%) of the title compound that was dried in a vacuum. Analysis: Calcd for C₁₄H₃₂MoN₂O₄S₄: C, 31.293; H, 7.003; N, 6.082. Found: C, 30.85; H, 6.89; N, 6.11.

The 2-(methoxy)ethyltrimethylammonium chloride was prepared by combining 1-chloro-2-methoxyethane (3.636 g, 38.5 mmol, see below in this example for synthesis) in a 25 ml round bottom flask with trimethyl amine (40% in water, 7.77 ml, 50 mmol). The mixture was heated at reflux for 24 hours, cooled to room temperature, and washed three times with 15 ml diethyl ether. The solvent was evaporated from the aqueous layer leaving a clumpy white solid that was washed with isopropanol and dried under vacuum to afford 4.136 g (70%) of the compound.

The 1-chloro-2-methoxyethane was prepared by combining 20 ml of chloroform with 2-methoxyethanol (5.4 ml, 69 mmol) in a nitrogen atmosphere. Thionyl chloride (5.0 ml, 69 mmol) and pyridine (5.5 ml, 69 mmol) were added sequentially by syringe, stirred at room temperature for 1 hour, and refluxed for 1 additional hour. The reaction components were cooled, quenched with water, washed twice with 25 ml 1M HCl, and the solvent was evaporated. The reaction products were filtered over silica gel and eluted with acetone to afford 5.94 g (91%) of the light brown component.

The mechanism for the synthesis of bis[2-(methoxy)ethyltrimethylammonium] tetrathiomolybdate according to the present example, is as follows:

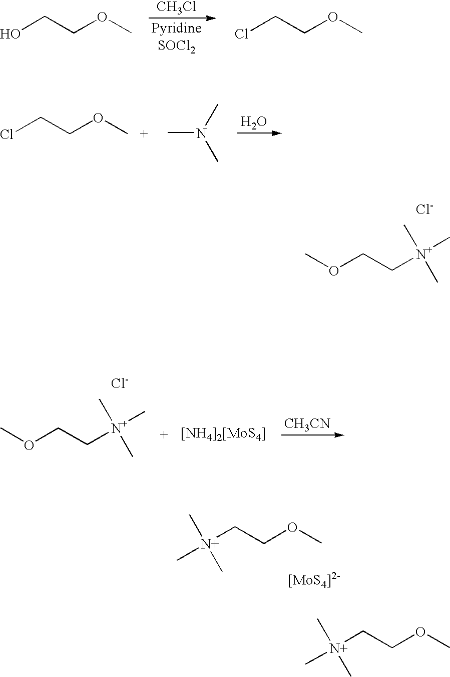

### 5.8 Example 8

Tetrathiomolybdate, bis[alkyldimethyl(phenylmethyl) ammonium]

This compound was prepared from ammonium tetrathiomolybdate (1.00 g, 3.84 mmol) and benzalkonium chloride (2.90 g, 8.07 mmol) according to the procedure of Example 6 and afforded 2.75 g (82%) of the title compound as a thick, red oil. IR (film, cm⁻¹) 471; ¹H NMR (300 MHz, DMSO-d6) δ7.58-7.46 (m, 10H), 4.54 (s, 4H), 3.24-3.20 (m, 4H), 2.95 (s, 12H), 1.83-1.71 (m, 4H), 1.30-1.21 (m, 40H), 0.88-0.81 (m, 6H); ES MS m/z [dodecyldimethyl(phenylmethyl) ammonium]⁺ 304.7, [tetradecyldimethyl(phenylmethyl) ammonium]⁺ 332.7; UV (DMSO) 473.5 nm (ε=10100).

### 5.9 Example 9

Tetrathiomolybdate, bis(1-ethyl-3-methyl-1H-imidazolium)

This compound was prepared from tetrathiomolybdate, bis(ammonium) (1.00 g, 3.84 mmol) and 1-ethyl-3-methyl-1H-imidazolium chloride (1.18 g, 8.06 mmol) according to the procedure of Example 2 with the following modifications; the mixture was filtered and the filtrate was concentrated in vacuo and the resulting solids were filtered, rinsed with EtOH, and ethyl ether and dried in a high vacuum desiccator for 24 hr giving the title compound (0.419 g, 24%) as a reddish-brown solid. IR (KBr pellet, cm⁻¹) 3064, 1566, 1167, 465; ¹H NMR (300 MHz, DMSO-d₆) δ9.24 (s, 1 H), 7.77 (s, 1 H), 7.69 (s, 1 H), 4.20 (q, J=7.3 Hz), 3.86 (s, 3 H), 1.40 (t, J=7.3 Hz); ¹³C NMR (75 MHz, DMSO-d₆) δ136.9, 123.5, 121.8, 44.1, 35.7, 15.3; MS m/z (C₆H₁₁N₂)⁺ 111.2; UV (H₂O) 468 nm (ε=11016). Analysis: Calcd for C₁₂H₂₂N₄ MoS₄: C, 32.28; H, 4.97; N, 12.55; S, 28.72. Found: C, 31.88; H, 4.87; N, 12.56; S, 28.79.

### 5.10 Example 10

Tetrathiomolybdate, bis(phenyltrimethylammonium)

This compound was prepared from tetrathiomolybdate, bis(ammonium) (1.00 g, 3.84 mmol) and phenyltrimethylammonium chloride (1.38 g, 8.06 mmol) according to the procedure of Example 2, and provided the title compound (0.859 g, 45%) as an orange solid. IR (KBr pellet, cm⁻¹) 3032, 3005, 1485, 1458, 473; ¹H NMR (300 MHz, DMSO-d₆) δ7.98 (m, 4 H), 7.61 (m, 6 H), 3.62 (s, 18 H); ¹³C NMR (75 MHz, DMSO-d₆) δ147.2, 130.0, 129.9, 120.5, 56.3; MS m/z (C₉H₁₄N)⁺ 136.2; UV (H₂O) 468 nm (ε=12900). Analysis: Calcd for C₁₈H₂₈N₂MoS₄: C, 43.53; H, 5.68; N, 5.64; S, 25.83. Found: C, 43.66; H, 5.98; N, 5.76; S, 25.73.

### 5.11 Example 11

Tetrathiomolybdate, bis(benzyltrimethylammonium)

This compound was prepared from tetrathiomolybdate, bis(ammonium) (0.500 g, 1.92 mmol) and benzyltrimethylammonium hydroxide (1.60 g of a 40% aqueous solution, 3.84 mmol) according to the procedure of Example 1, providing the title compound (0.869 g, 91%) as a red solid. Mp 179-181° C. (decomp); IR (KBr pellet, cm⁻¹) 2995, 1483, 1454, 474; ¹H NMR (300 MHz, DMSO-d₆) δ7.52 (m, 10 H), 4.56 (s, 4 H), 3.04 (s 18 H); ¹³C NMR (75 MHz, DMSO-d₆) δ132.9, 130.2, 128.9, 128.4, 67.7, 51.7; MS m/z (C₁₀H₁₆N)⁺ 150.3 ; UV (H₂O) 468 nm (ε=12008). Analysis: Calcd for C₂₀H₃₂N₂MoS₄: C, 45.78; H, 6.15; N, 5.34; S, 24.44. Found: C, 45.67; H, 6.15; N, 5.62; S, 24.41.

### 5.12 Example 12

Tetrathiomolybdate, pentane-1,5-bis(trimethylammonium)

To a suspension of tetrathiomolybdate, bis(ammonium) (0.100 g, 0.226 mmol) in CH₃CN (2 mL) was added a solution of pentane-1,5-bis(trimethyl ammonium) iodide (0.054 g, 0.205 mmol) dissolved in water (2 mL). After stirring for 5 hours at room temperature, the brownish-red solids were filtered, suspended in water then refiltered and washed with EtOH, iPrOH and Et₂O. After drying in a high-vacuum desiccator overnight a red solid was obtained giving the title compound (0.051 g, 60%). IR (KBr pellet, cm⁻¹) 2997, 472; ¹H NMR (300 MHz, MeOH-d₅) δ3.22 (m, 4 H), 2.98 (s 18 H), 1.70 (m, 4 H), 1.31 (2 H); ¹³C NMR (75 MHz, DMSO-d₆) δ79.4, 64.9, 52.2, 21.6; UV (H₂O) 468 nm (ε=12025). Analysis: Calcd for C₁₁H₂₈N₂MoS₄: C, 32.03; H, 6.84; N, 6.79; S, 31.09. Found: C, 32.40; H, 6.74; N, 6.80; S, 30.87.

### 5.13 Example 13

Tetrathiomolybdate, bis(2-hydroxyiminomethyl-1-methyl-pyridinium)

This compound was prepared from tetrathiomolybdate, bis(ammonium) (1.00 g, 3.84 mmol) and 2-hydroxyiminomethyl-1-methyl-pyridinium chloride (1.39 g, 8.06 mmol) according to the procedure of Example 2 with the following modification: water (10 mL) was added to dissolve the ammonium halide. The title compound (1.20 g, 66%) was provided as a red solid. IR (KBr pellet, cm⁻¹) 1002, 477; ¹H NMR (300 MHz, DMSO-d₆) δ9.05 (app d, 1 H), 8.67 (s, 1 H), 8.54 (app t, 1 H), 8.08 (app d, 1 H), 8.04 (app t, 1 H), 4.39 (s, 3 H); ¹³C NMR (75 MHz, DMSO-d₆) δ147.6, 147.1, 145.1, 142.0, 127.4, 124.9, 46.3; MS m/z (C₇H₉N₂O)⁺ 137.1; UV (H₂O) 468 nm (ε=12092).

### 5.14 Example 14

Tetrathiomolybdate, bis(1,1-dimethylpyrrolidinium)

This compound was prepared from ammonium tetrathiomolybdate (0.644 g, 2.47 mmol) and 1,1-dimethylpyrrolidinium iodide (1.18 g, 5.19 mmol) according to the procedure of Example 2, and provided an orange solid. IR (KBr, cm⁻¹) 471; ¹H NMR (300 MHz, DMSO-d6) δ2.09 (m, 4H), 3.10 (s, 6H), 3.47 (t, J=6 Hz, 4H); ¹³C (75 MHz, DMSO-d6) δ21.44, 50.86, 64.68; ES MS m/z (1,1-dimethylpyrrolidinium)⁺ 100; UV (H₂O) 468 nm (ε=14130). Analysis: Calcd for C₁₂H₂₈N₂MoS₄: C, 33.94; H, 6.64; N, 6.59; S, 30.21. Found: C, 33.68; H, 6.97; N, 6.58; S, 30.06.

### 5.15 Example 15

Tetrathiomolybdate, ethylene bis ammonium

Ammonium chloride (0.411 g, 7.68 mmol) and ethylene diamine (0.230 g, 3.84 mmol) were dissolved in 50 mL of water. To this solution was added ammonium tetrathiomolybdate (1.0 g, 3.84 mmol) and the mixture was stirred for 3 hours. The brick red solid that formed was collected by filtration (233 mg), rinsed with isopropanol and Et₂O and dried under high vacuum for 24 hours in a vacuum desiccator in the presence of P₂O₅. The mother liquors were concentrated to ˜⅓ their original volume and the newly formed crystals were again collected by filtration and rinsed (387 mg). Total recovery of the title compound was 620 mg, (56.8%). IR (KBr, cm⁻¹) 474; ¹H NMR (300 MHz, DMSO-d6) δ3.08 (s, 4H); 7.79 (br s, 4H); ¹³C (75 MHz, DMSO-d6) δ37.08; ES MS m/z (ethylene bis ammonium)⁺ 60.5; UV (H₂O) 468 nm (ε=12310). Analysis: Calcd for C₂H₁₀MoN₂S₄: C, 8.39; H, 3.52; N, 9.78; S, 44.80. Found: C, 8.75; H, 3.28; N, 10.05; S, 44.99.

### 5.16 Example 16

Tetrathiomolybdate, bis(1,4-dimethylpyridinium)

This compound was prepared from ammonium tetrathiomolybdate (0.50 g, 1.92 mmol) and 1,4-dimethyl pyridinium iodide (0.95 g, 4.03 mmol) according to the procedure of Example 2 and provided 203 mg (24%) of the title compound as an orange solid. IR (KBr, cm⁻¹) 469; ¹H NMR (300 MHz, DMSO-d6) δ2.49 (s, 3H), 4.30 (s, 3H), 7.93, (d, J=6 Hz, 2H), 8.85 (d, J=6 Hz, 2H); ¹³C (75 MHz, DMSO-d-6) δ21.31, 47.03, 127.94 (2C), 144.77, 157.93; ES MS miz bis(1,4-dimethylpyridinium)⁺ 108.2 ; UV (H₂O) 468 nm (ε=13480). Analysis: Calcd for C₁₄H₂₀N₂MoS₄: C, 38.17; H, 4.57; N, 6.35; S, 29.11. Found: C, 38.27; H, 4.24; N, 6.36; S, 28.89.

### 5.17 Example 17

Tetrathiomolybdate, bis(phenyltrimethyl ammonium)

This compound was prepared according to the procedure of Example 1. IR (cm−1) 470; ¹H NMR (400 MHz, DMSO-d6) δ7.99-7.97 (d, J=8.4 Hz, 4H), 7.66-7.56 (m, 6H), 3.62 (s, 18H); UV (H₂O) 468 nm (ε=13151). Analysis: Calcd for C₁₈H₂₈MoN₂S₄: C, 43.53; H, 5.68; N, 5.64; S, 25.83. Found: C, 42.72; H, 5.20; N, 5.27; S, 27.54.

### 5.18 Example 18

Tetrathiomolybdate, bis(vinyltrimethyl ammonium)

This compound was prepared according to the procedure of Example 1. IR (cm−1) 470; ¹H NMR (400 MHz, DMSO-d6) δ6.62-6.56 (m, 2H), 5.76 (d, J=15.2 Hz, 2H), 5.54 (m, 2H), 3.24 (s, 18H); UV (H₂O) 468 nm (ε=18012).

### 5.19 Example 19

Tetrathiomolybdate, bis(cyclopropylmethyltrimethyl ammonium)

This compound was prepared according to the procedure of Example 2. IR (cm−1) 474; ¹H NMR (400 MHz, DMSO-d6) δ3.24 (d, J=7.2Hz, 4H), 3.11 (s, 18H), 1.16 (m, 2H), 0.72-0.69 (m, 4H), 0.42-0.38 (m, 4H); UV (H₂O) 468 nm (ε=14239).

### 5.20 Example 20

Tetrathiomolybdate, bis(benzylphenyldimethylammonium)

This compound was prepared according to the procedure of Example 2. IR (cm⁻¹) 470; ¹H NMR (400 MHz, DMSO-d6) δ7.76-6.60 (m), 5.00 (s), 2.87 (s); UV (H₂O) 468 nm (ε=15606).

### 5.21 Example 21

Tetrathiomolybdate, hexane-1,6-bis(trimethyl ammonium)

This compound was prepared according to the procedure of Example 2. IR (cm⁻¹) 478; UV (DMSO) 468 nm (ε=12666).

### 5.22 Example 22

Tetrathiomolybdate, bis[(2-hydroxyethyl)trimethyl ammonium]

Choline hydroxide (50% w/w in water, 2.56 mL, 11.3 mmol) was added to a solution of ammonium molybdate tetrahydrate (1.0 g, 5.66 mmol of Mo) in 2.5 mL of deionized water. Hydrogen sulfide gas was bubbled through the solution for 50 minutes at room temperature, during which time the color of the solution turned from brown to red. Nitrogen gas was bubbled through the solution for 10 min to purge the reaction mixture of dissolved hydrogen sulfide and the solvent was removed under reduced pressure. The red solid was dissolved in deionized water (3×25 mL) and the solvent was removed repeatedly under reduced pressure to remove dissolved ammonia. The crude product was dissolved in 20 mL of deionized water and filtered. The product was precipitated by addition of isopropanol to the aqueous layer. The solids were collected by filtration and washed with ethanol (3×) and diethyl ether (3×) to produce the crude title compound (2.218 g, 90%). Recrystallization from deionized water and isopropanol, and washing with ethanol (3×) and diethyl ether (3×) afforded the title compound (1.565 g, 62%) as plate-like red crystals: ¹H NMR (300 MHz, DMSO-d₆) δ5.23 (br t, J=4.7 Hz, 2H), 3.85 (br s, 4H), 3.46-3.43 (m, 4H), 3.14 (s, 18H).

### 5.23 Example 23

Tetrathiomolybdate, bis[(2-hydroxyethyl)trimethyl ammonium]

Hydrogen sulfide gas was bubbled through a solution of choline hydroxide (50% w/w in water, 5.2 mL, 23 mmol) and ammonium hydroxide (30% w/w in water, 8.2 mL, 70 mmol) in a tared pressure vessel for six minutes. The amount of hydrogen sulfide dissolved in the solution was 2.5 g. Ammonium molybdate tetrahydrate (2.01 g, 11.4 mmol) dissolved in deionized water (2.5 mL) was added to the pressure vessel and the sides washed with deionized water (0.5 mL). The reaction mixture was sealed and stirred at room temperature for 2 hours and 45 minutes, during which time crystals formed in the red solution. The mixture was transferred to a round-bottomed flask and deionized water added until the solid dissolved. The solvent was removed under reduced pressure, the residue taken up in deionized water, and the concentration step repeated. The resulting red residue was recrystallized from deionized water and isopropanol and the red plates washed once with isopropanol, once with ethanol, once with diethyl ether, and dried in a vacuum desiccator in the presence of phosphorus pentoxide for 23 hours to afford 3.05 g (62%) of the title compound. ¹H NMR (300 MHz, DMSO-d6) δ5.21 (t, J=4.6 Hz, 2H), 3.87-3.79 (m, 4H), 3.43 (t, J=4.6 Hz, 4H), 3.13 (s, 18H); ¹³C NMR (75 MHz, DMSO-d₆) δ67.1 (2C), 55.5 (2C), 53.3 (6C); MS m/z (choline)⁺ 104.2; UV (H₂O) 468 nm (ε=12400). Analysis: Calcd for C₁₀H₂₈MoN₂O₂S₄: C, 27.77; H, 6.52; N, 6.48; S, 29.65. Found: C, 27.76; H, 6.59; N, 6.85; S, 29.52.

### 5.24 Example 24

Tetrathiomolybdate, bis[(2-hydroxyethyl)trimethyl ammonium]

Deionized water (200 mL) was added to choline hydroxide (50% w/w in water, 38.2 mL, 14.7 mmol), followed by ammonium tetrathiomolybdate (19.1 g, 7.35 mmol) and the flask swirled until all the solid material was dissolved. The solution was placed on a rotary evaporator under vacuum (ca. 5-10 torr) with the bath at 20° C. for 110 minutes and the water was replaced as needed to maintain a constant volume. The reaction mixture was filtered to remove small amounts of solid impurities, and the product was precipitated from the filtrate with isopropanol (1 L). The solid was collected by filtration, washed with isopropanol, ethanol, and diethyl ether and then dried under high vacuum for 21 hours to afford 28.32 g (89%) of the desired product as an orange powder. The crude product was recrystallized from deionized water and isopropanol to afford red plates, which were washed once with isopropanol, once with ethanol, once with diethyl ether, and dried in a vacuum desiccator in the presence of phosphorus pentoxide for 24 hours to afford 20.76 g (65%) the title compound. ¹H NMR (300 MHz, DMSO-d₆) δ5.23 (t, J=4.8 Hz, 2H), 3.89-3.81 (m, 4H), 3.44 (t, J=4.8 Hz, 4H), 3.14 (s, 18H). Analysis: Calcd for C₁₀H₂₈MoN₂O₂S₄: C, 27.77; H, 6.52; N, 6.48; S, 29.65. Found: C, 27.91; H, 6.21; N, 6.26; S, 29.70.

### 5.25 Example 25

Tetrathiomolybdate, bis[(2-hydroxyethyl)trimethyl ammonium]

In a 500 mL Erlenmeyer flask, [NH₄]₂[MoS₄] (100 g, 0.380 mol) was suspended in 200 mL of distilled water. To the suspension was added a 45 wt % solution of choline hydroxide in methanol (250 mL) (commercially available). Upon addition, a large amount of a bright red precipitate formed. Under a stream of nitrogen the suspension was heated to 40° C. with vigorous stirring until all solids dissolved (˜1 hour). The deep red solution was kept under reduced pressure for 4 hours, during which time a red crystalline solid precipitated. The suspension was cooled in an ice bath for 30 minutes and then filtered. The product was washed with isopropyl alcohol until the washings were clear, then washed with diethyl ether and finally dried under vacuum to give 138 g of highly crystalline product (83% yield).

### 5.26 Example 26

X-Ray Structure Determination of Choline Tetrathiomolybdate

Diffraction data for a red crystal of (Choline)₂MoS₄ were collected at 158(2) K using a Siemens SMART area diffractometer (Siemens AG, Wittelsbacherplatz 2, D-80333, Munichm Federal Republic of Germany). A monoclinic P lattice was obtained with a cell: a=18.6066(1), b=12.7061(1), c=17.7621(2), and β=117.540(1) (Space group P2₁/c). The crystal structure was solved by direct methods and shows 2 [MoS₄] and 4 choline cations in the asymmetric unit. After least squares refinement of all non-hydrogen atoms in the unit cell and convergence the final R factor was 0.06. The cell coordinates of the atoms in the unit cell are as follows:

| Atom | x/a | y/b | z/c |
|---|---|---|---|
| Mo1 | 0.3502 | 0.7432 | 0.4307 |
| Mo2 | 0.1641 | 0.2579 | 0.0786 |
| N1 | 0.4724 | 0.6022 | 0.2250 |
| N2 | 0.3306 | 0.3 142 | 0.4043 |
| N3 | −0.0120 | 0.0317 | −0.3170 |
| N4 | 0.1920 | 0.8572 | 0.1184 |
| S1 | 0.2450 | 0.8268 | 0.4246 |
| S2 | 0.4354 | 0.8566 | 0.4245 |
| S3 | 0.4096 | 0.6553 | 0.5492 |
| S4 | 0.3114 | 0.6324 | 0.3236 |
| S5 | 0.2769 | 0.1760 | 0.1066 |
| S6 | 0.1167 | 0.1908 | 0.1604 |
| S7 | 0.0768 | 0.2372 | −.0535 |
| S8 | 0.1883 | 0.4248 | 0.1079 |
| O1 | 0.6860 | 0.9215 | 0.6690 |
| O2 | 0.6133 | 0.6075 | 0.8536 |
| O3 | 0.895i | 0.3408 | 0.7335 |
| O4 | 0.9355 | 0.3376 | 0.5362 |
| C1 | 0.7156 | 0.8812 | 0.6189 |
| C2 | 0.6576 | 0.7959 | 0.5590 |
| C3 | 0.6471 | 0.6801 | 0.6669 |
| C4 | 0.7534 | 0.6488 | 0.6274 |
| C5 | 0.6139 | 0.6151 | 0.5265 |
| C6 | 0.6288 | 0.5111 | 0.8973 |
| C7 | 0.6144 | 0.4157 | 0.8417 |
| C8 | 0.5016 | 0.4766 | 0.7046 |
| C9 | 0.5221 | 0.2904 | 0.7373 |
| C10 | 0.4710 | 0.4023 | 0.8135 |
| C11 | 0.9086 | 0.4169 | 0.7986 |
| C12 | 0.9240 | 0.5165 | 0.7797 |
| C13 | 1.0580 | 0.4580 | 0.7905 |
| C14 | 1.0186 | 0.6375 | 0.7813 |
| C15 | 1.0496 | 0.5430 | 0.9103 |
| C16 | 0.9086 | 0.4815 | 0.5675 |
| C17 | 0.8505 | 0.4262 | 0.4598 |
| C18 | 0.7199 | 0.3849 | 0.3353 |
| C19 | 0.8417 | 0.3763 | 0.3214 |
| C20 | 0.8182 | 0.2446 | 0.4035 |

### 5.27 Example 27

Chemical Stability Data for Analogs

| Salt | MW | |
|---|---|---|
| | | % remaining |
| | | After 10 days |
| | | Oven to room |
| | | conditions |
| propane-1,3-bis(trimethylammonium) | 384.5 | 52 |
| 1-ethyl-3-methyl-1H-imidazolium | 446.5 | 75 |
| Trimethylphenylammonium | 496.6 | 85 |
| Tetrapropylammonium | 596.92 | *82 |
| Ammonium | 260.28 | *48 |
| Acetylcholine | 516.6 | 39 |
| Choline | 432.52 | 62 |
| Triethylphenylammonium | 580.77 | 76 |
| Methyltriethylammonium | 456.63 | 63 |
| 1,1-dimethylpyrrolidinium | 424.57 | 70 |
| butane-1,4-bis(trimethylammonium) | 398.53 | 40 |
| 1,4-dimethylpyridinium | 440.53 | 32 |
| Trimethylbenzylammonium | 524.69 | 94 |
| Ethylenebis(trimethylammonium) | 370.5 | 40 |
| pentane-1,5-bis(trimethylammonium) | 412.5 | 86 |
| | | 7 days, |
| | | 74-77% RH, RT |
| bis(phenyltrimethylammonium) | 496.61 | 98 |
| bis(vinyltrimenthyammonium) | 396.49 | 72 |
| bis(cyclopropylmethyltrimethylammonium) | 452.60 | 86 |
| bis(benzylphenyldimethylammonium) | 648.81 | 45 |
| hexane-1,6-bis(trimethylammonium) | 426.56 | 94 |

*9 days; different study

### 5.28 Example 28

Biological Data Comparing Tetrathiomolybdate with Choline Tetrathiomolybdate In Vivo 3LL Lewis Lung Carcinoma: Primary Tumor Growth

The tumor model used was 3LL JF in C57BL/6 mice. This tumor line arose spontaneously in 1951 as carcinoma of the lung in a C57BL/6 mouse (Cancer Res. 15:39, 1955. See, also Malave et al., J. Nat'l. Canc. Inst. 62:83-88 (1979). It is propogated by passage in C57BL/6 mice by subcutaneous inoculation and is tested in semiallogeneic C57BL/6×DBA/2 F₁ mice or in allogeneic C3H mice. Typically six animals per group for subcutaneously implant, or ten for intramuscular implant are used. Tumor may be implanted subcutaneously as a 2-4 mm fragment, or intramuscular or intramuscular as an inoculum of suspended cells of about 0.5-2×10⁶-cells. Treatment begins 24 hours after implant or is delayed until a tumor of specified size (usually approximately 100 mg) can be palpated. The test compound is administered daily for 11-25 days.

Animals are followed by weighing, palpation and measurement of tumor size. Typical tumor weight in untreated control recipients on day 12 after subcutaneous inoculation is 500-2500 mg and typical median survival time is 18-28 days. A positive control compound, for example cyclophosphamide at 170 mg/kg/injection is given every 6 days. Results computed include mean animal weight, tumor size, tumor weight and survival time. For confirmed therapeutic activity, the test composition should be tested in two multi-dose assays. Female C57/BL mice were inoculated with 1×10⁶ Lewis Lung carcinoma (3LL) cells subcutaneously in the middle of the back. Treatment with tetrathiomolybdate and choline thiomolybdate was initiated the day after tumor inoculation (50 mg/kg po by gavage). Cyclophosphamide (170 mg/kg given every 6 days subcutaneously beginning on day 3 after tumor cell inoculation) was used as a control. Tumor volumes and animal weights were measured 2×/week and the animals euthanized when the volume of the tumors in the control group reached an average of 2000 mm³. The ratio (T/C) of the tumor volumes in the treated group (T) to the control group (C) were determined. Terminal cardiac bleeds were obtained and analyzed for red blood cell concentration (hematocrit, HCT).

| | | | MOUSE | |
|---|---|---|---|---|
| | | | WEIGHT % | |
| TREATMENT | N | T/C | of initial | HCT** |
| Water | 19 | 1 | 106% | 35% |
| TM | 17 | 0.59 | 93%* | 35% |
| CHTM | 17 | 0.54 | 103% | 35% |
| Cyclophosphamide | 5 | 0.74 | 105% | 40% |

*significant;
**normal is 45%

### 5.29 Example 29

Matrigel Plug Assay

This assay is performed essentially as described by Passaniti et al., Lab Invest. 67:519-528 (1992). Ice-cold Matrigel® (e.g., 500 μL) (Collaborative Biomedical Products, Inc., Bedford, Mass.) is mixed with heparin (e.g., 50 μg/ml), FGF-2 (e.g., 400 ng/ml) and the compound to be tested. In some assays, bFGF may be substituted with tumor cells as the angiogenic stimulus. The Matrigel® mixture is injected subcutaneously into 4-8 week-old athymic nude mice at sites near the abdominal midline, preferably 3 injections per mouse. The injected Matrigel® forms a palpable solid gel. Injection sites are chosen such that each animal receives a positive control plug (such as FGF-2+heparin), a negative control plug (e.g., buffer+heparin) and a plug that includes the compound being tested for its effect on angiogenesis, e.g., (FGF-2+heparin+compound). All treatments are preferably run in triplicate. Animals are sacrificed by cervical dislocation at about 7 days post injection or another time that may be optimal for observing angiogenesis. The mouse skin is detached along the abdominal midline, and the Matrigel® plugs are recovered and scanned immediately at high resolution. Plugs are then dispersed in water and incubated at 37° C. overnight. Hemoglobin levels are determined using Drabkin's solution (e.g., obtained from Sigma) according to the manufacturers' instructions. The amount of Hb in the plug is an indirect measure of angiogenesis as it reflects the amount of blood in the sample. In addition, or alternatively, animals may be injected prior to sacrifice with a 0.1 ml buffer (preferably PBS) containing a high molecular weight dextran to which is conjugated as a fluorophore. The amount of fluorescence in the dispersed plug, determined fluorimetrically, also serves as a measure of angiogenesis in the plug. Staining with mAb anti-CD31 (CD31 is “platelet-endothelial cell adhesion molecule or PECAM”) may also be used to confirm neovessel formation and microvessel density in the plugs.

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims. All publications and patents cited herein are incorporated by reference.

What is claimed is:

1. A compound of structural formula (I): 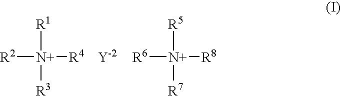
or a solvate or hydrate thereof wherein:
R¹, R², R³, R⁵, R⁶ and R⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl;
R⁴ and R⁸ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl or are absent when N is part of an aromatic ring;
optionally, R¹ and R² taken together are alkyldiyl, substituted alkyldiyl, heteroalkyldiyl or substituted heteroalkyldiyl;
optionally, R⁵ and R⁶ taken together are alkyldiyl, substituted alkyldiyl, heteroalkyldiyl or substituted heteroalkyldiyl;
optionally, R¹ and R² taken together, R² and R³ taken together and R² and R⁴ taken together are alkyldiyl, substituted alkyldiyl, heteroalkyldiyl or substituted heteroalkyldiyl;
optionally, R⁵ and R⁶ taken together, R⁶ and R⁷ taken together and R⁶ and R⁸ taken together are alkyldiyl, substituted alkyldiyl, heteroalkyldiyl or substituted heteroalkyldiyl;
optionally, R³ and R⁷ taken together are alkyldiyl, substituted alkyldiyl, heteroalkyldiyl or substituted heteroalkyldiyl; and
Y⁻² is (MoS₄)⁻², (Mo₂S₁₂)⁻², (Mo₂S₉)⁻², (Mo₂S₇)⁻², (Mo₂S₈)⁻², (Mo₂S₁₁)⁻², (Mo₂S₆)⁻² or (Mo₂S₁₃)⁻²;
with the proviso that if Y is (MoS₄)⁻² and R¹, R², R³, R⁴, R⁵, R⁶, R⁷ and R⁸ are identical then each of R¹, R², R³, R⁴, R⁵, R⁶, R⁷ and R⁸ is not hydrogen, methyl, ethyl or n-propyl.

2. The compound of claim 1, wherein Y is (MoS₄)⁻².

3. The compound of claim 1, wherein

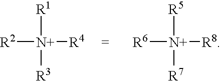

4. The compound of claim 3, wherein Y is (MoS₄)⁻².

5. The compound of any one of claims 1-4, wherein at least one of R¹, R², R³ and R⁴ is not alkyl.

6. The compound of claim 1 or 2, wherein at least one of R¹, R², R³, R⁴, R⁵, R⁶, R⁷ and R⁸ is not alkyl.

7. The compound of any one of claims 1-4, wherein R¹, R² and R⁴ are hydrogen, alkanyl or substituted alkanyl.

8. The compound of any one of claims 1-4, wherein R¹, R² and R⁴ are hydrogen, methyl or ethyl.

9. The compound of any one of claims 1-4, wherein R¹ and R² are alkanyl.

10. The compound of any one of claims 1-4, wherein R¹ and R² are methyl or ethyl.

11. The compound of any one of claims 1-4, wherein R¹ is alkanyl, substituted alkanyl, alkenyl, substituted alkenyl, aryl, substituted aryl, arylalkyl, substituted arylalkyl, cycloalkyl or substituted cycloalkyl.

12. The compound of any one of claims 1-4, wherein R¹ and R² taken together are alkyleno, substituted alkyleno, heteroalkyleno or substituted heteroalkyleno.

13. The compound of any one of claims 1-4, wherein R¹ and R² taken together are alkyleno or heteroalkyleno.

14. The compound of any one of claims 1-4, wherein R¹ and R² taken together, R² and R³ taken together and R² and R⁴ taken together are alkyleno, substituted alkyleno, heteroalkyleno or substituted heteroalkyleno.

15. The compound of any one of claims 1-4, wherein R¹ and R² taken together, R² and R³ taken together and R² and R⁴ taken together are alkyleno.

16. The compound of any one of claims 1-4, wherein R¹(R²)(R³)(R⁴)N has the structure:

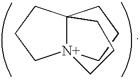

17. The compound of any one of claims 1-4, wherein R³ and R⁷ taken together are alkyleno, substituted alkyleno, heteroalkyleno or substituted heteroalkyleno.

18. The compound of any one of claims 1-4, wherein R³ and R⁷ taken together are alkyleno or heteroalkyleno.

19. The compound of any one of claims 1-4, wherein R¹, R² and R⁴ are hydrogen, alkanyl or substituted alkanyl and R³ is alkyl, substituted alkyl, alkenyl, aryl, arylalkyl, cycloalkyl or R³ and R⁷ taken together are alkyleno, substituted alkyleno, heteroalkyleno or substituted heteroalkyleno.

20. The compound of any one of claims 1-4, wherein R¹, R² and R⁴ are methyl or ethyl and R³ is alkyl, substituted alkyl, alkenyl, aryl, arylalkyl, cycloalkyl or R³ and R⁷ taken together are alkyleno or heteroalkyleno.

21. The compound of any one of claims 1-4, wherein R¹, R² and R⁴ are methyl or ethyl and R³ is alkyl, substituted alkyl, alkenyl, aryl, arylalkyl or cycloalkyl.

22. The compound of any one of claims 1-4, wherein R¹(R²)(R³)(R⁴)N has the structure:

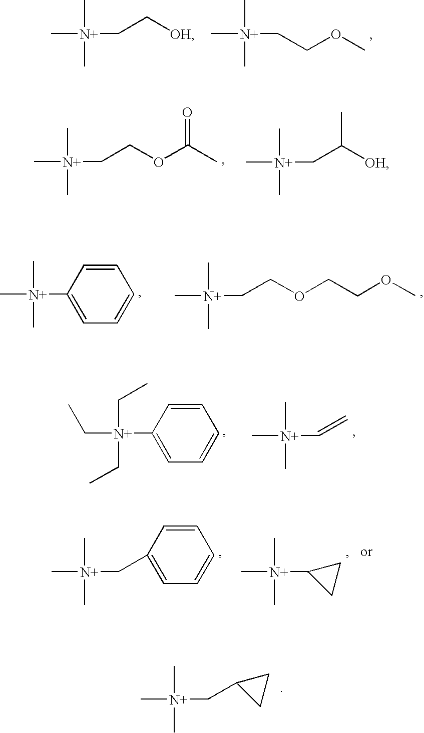

23. The compound of any one of claims 1-4, wherein R¹(R²)(R³)(R⁴)N has the structure:

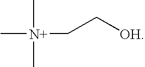

24. The compound of any one of claims 1-4, wherein R¹(R²)(R³)(R⁴)N has the structure:

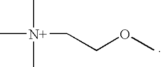

25. The compound of any one of claims 1-4, wherein R¹(R²)(R³)(R⁴)N has the structure:

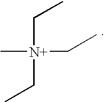

26. The compound of any one of claims 1-4, wherein R¹, R² and R⁴ are methyl or ethyl and R³ and R⁷ taken together are alkyleno or heteroalkyleno.

27. The compound of any one of claims 1-4, wherein R¹(R²)(R³)(R⁴)N has the structure:

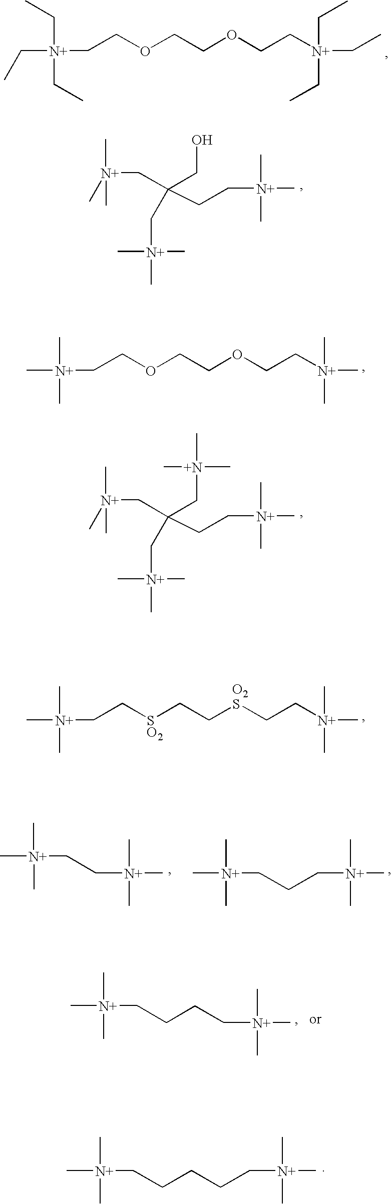

28. The compound of any one of claims 1-4, wherein R¹, R² and R⁴ are hydrogen and R³ is substituted alkyl, cycloalkyl or substituted heteroaryl or R³ and R⁷ taken together are alkyleno.

29. The compound of any one of claims 1-4, wherein R¹ and R² are alkanyl and R³ and R⁴ are hydrogen, alkyl, substituted alkyl, aryl, arylalkyl or alkyleno.

30. The compound of any one of claims 1-4, wherein R¹ and R² are methyl or ethyl and R³ and R⁴ are hydrogen, alkyl, substituted alkyl, aryl, arylalkyl or alkyleno.

31. The compound of any one of claims 1-4, wherein R¹(R²)(R³)(R⁴)N has the structure: 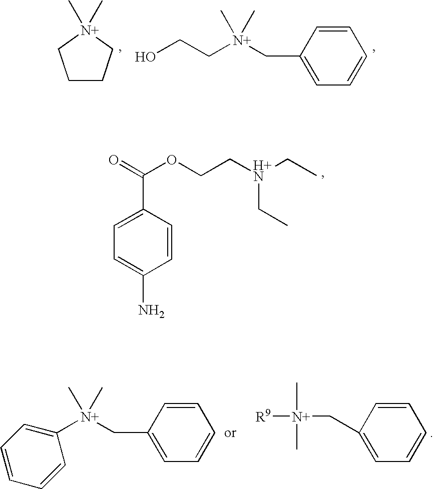
wherein R⁹ is a mixture of straight chain alkanyl groups which have at least eight carbon atoms and not more than eighteen carbon atoms.

32. The compound of any one of claims 1-4, wherein R¹, R² and R⁴ are hydrogen and R³ is substituted alkyl, substituted heteroaryl, cycloalkyl or alkyleno.

33. The compound of any one of claims 1-4, wherein R¹(R²)(R³)(R⁴)N has the structure:

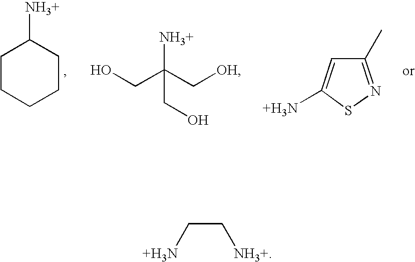

34. The compound of any one of claims 1-4, wherein R¹ and R² taken together are alkyleno, substituted alkyleno, heteroalkyleno or substituted heteroalkyleno, R³ is alkyl or substituted alkyl and R⁴ is hydrogen or is absent.

35. The compound of any one of claims 1-4, wherein R¹(R²)(R³)N or R¹(R²)(R³)(R⁴)N has the structure:

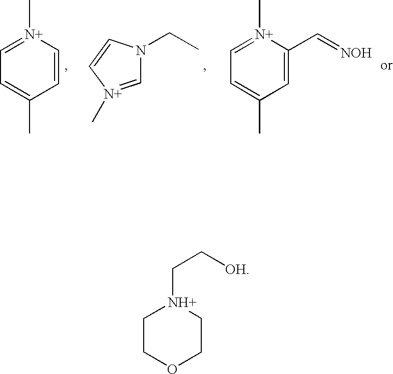

36. A pharmaceutical composition comprising a compound of any one of claims 1-4 and a pharmaceutically acceptable diluent, excipient or adjuvant.

37. A method for treating or preventing cancer in a patient comprising administering to the patient in need of such treatment a therapeutically effective amount of a compound of any one of claims 1-4.

38. A method for treating or preventing cancer in a patient comprising administering to the patient in need of such treatment a therapeutically effective amount of the pharmaceutical composition of claim 35.

39. The method of claim 36 further comprising administering to the patient in need of such treatment a therapeutically effective amount of another anti-cancer agent or a pharmaceutical composition comprising the other anti-cancer agent and a pharmaceutically acceptable diluent, excipient or adjuvant.

40. The method of claim 37 further comprising administering to the patient in need of such treatment a therapeutically effective amount of another anti-cancer agent or a pharmaceutical composition comprising the other anti-cancer agent and a pharmaceutically acceptable diluent, excipient or adjuvant.

41. The method of claim 36 further comprising administering to the patient in need of such treatment a therapeutically effective amount of zinc or a pharmaceutical composition of zinc.

42. The method of claim 37 further comprising administering to the patient in need of such treatment a therapeutically effective amount of zinc or a pharmaceutical composition comprising zinc and a pharmaceutically acceptable diluent, excipient or adjuvant.

43. The method of claim 36, wherein the cancer is breast cancer, renal cancer, brain cancer colon cancer, prostrate cancer, chondrosarcoma or angiosarcoma.

44. A method for treating or preventing wet type macular degeneration or rheumatoid arthritis in a patient comprising administering to the patient in need of such treatment a therapeutically effective amount of a compound of any one of claims 1-4.

45. A method for treating or preventing wet type macular degeneration or rheumatoid arthritis in a patient comprising administering to the patient in need of such treatment a therapeutically effective amount of the pharmaceutical composition of claim 35.

46. A method for treating or preventing aberrant vascularization in a patient comprising administering to the patient in need of such treatment a therapeutically effective amount of a compound of any one of claims 1-4.

47. A method for treating or preventing aberrant vascularization in a patient comprising administering to the patient in need of such treatment a therapeutically effective amount of a pharmaceutical composition of claim 35.

48. A method for treating or preventing excess copper levels in a patient comprising administering to the patient in need of such treatment a therapeutically effective amount of a compound of any one of claims 1-4.

49. A method for treating or preventing excess copper levels in a patient comprising administering to the patient in need of such treatment a therapeutically effective amount of a pharmaceutical composition of claim 35.
